(19) United States
(12) Patent Application Publication
(10) Pub. No.: US 2013/0096524 A1
(43) Pub. Date: Apr. 18, 2013

(54) WOUND TREATMENT ARTICLE, CONTAINING A PORTION OF MODIFIED NATURAL FIBERS OR SYNTHETIC FIBERS

(71) Applicant: Birgit Riesinger, Ostbevern (DE)

(72) Inventor: Birgit Riesinger, Ostbevern (DE)

(21) Appl. No.: 13/631,742

(22) Filed: Sep. 28, 2012

Related U.S. Application Data
(63) Continuation of application No. 13/042,441, filed on Mar. 7, 2011, now abandoned.
Application No. 13/042,441 is a continuation of application No. PCT/EP2009/061571, filed on Sep. 7, 2009.

(30) Foreign Application Priority Data
Sep. 5, 2008 (DE) 102008045833.3

(51) Int. Cl. A61F 13/00

(52) U.S. Cl. CPC A61F 13/00042; A61F 13/00017
USPC 604/369; 604/367; 604/375; 604/376

(57) ABSTRACT

The invention relates to a wound treatment item having a proportion of modified natural fibers or synthetic fibers with a high dehydrating capacity.

### CROSS-REFERENCE TO RELATED APPLICATIONS

This application is a continuation of U.S. Ser. No. 13/042,441, filed Mar. 7, 2011, which claims priority under 35 U.S.C. §120 and §365(c) to PCT International Application No. PCT/EP2009/061571, filed Sep. 7, 2009, designating the United States, which claims priority to German Application No. 10 2008 045 833.3, filed Sep. 5, 2008, all of which are incorporated herein by reference in their entireties.

### FIELD OF THE INVENTION

The invention relates to the field of wound treatment; more particularly, the invention relates to the field of wound treatment using an article having dehydration characteristics.

### BACKGROUND OF THE INVENTION

Conventional wound treatment articles, particularly wound dressings, consist in large part of fibers of natural origin, particularly cellulose and cellulose derivatives, since these materials are capable of absorbing fluids, particularly blood and exudate. Wound treatment articles of this kind often have synthetic polymers or fibers as well, which lend the articles particular properties. These may be, for example, a single-sided sheath made from a water-impermeable film, which serves to protect undergarments, or a film facing towards the wound and made from a polymer that does not adhere to the wound. When superabsorbent polymers are included in the core of a wound dressing, it is also endowed with particular properties, such as greater capacity to absorb wound fluids.

Conventional wound dressings of this type are known, for example, from German patents DE10059439, DE20207356, DE102007036758, DE102007036757, DE102007049430, DE102007049429, and DE102007 049428 already granted to the applicant of the present invention. Each of the aforementioned disclosures are hereby incorporated by reference into the present application.

Natural fibers in particular are associated with certain disadvantages. Natural fibers can be wetted and typically absorb moisture (water absorption capacity), but they do not retain it permanently. This behavior may cause, for example, wound macerations (softening and inflammation of the surrounding skin due to prolonged contact with a fluid) and may also hasten the growth of bacteria, since these wound treatment articles offer the damp (and often warm) microclimate such bacteria need. When they are wetted, natural fibers often lose their structural integrity as well, and therewith also their padding and heat insulating properties.

### SUMMARY OF THE INVENTION

The object of the present invention is therefore to provide a wound treatment article that does not have the abovementioned disadvantages of the products from the prior art. This object is attained with a wound treatment article as described in the present main claim; the subordinate claims describe preferred embodiments thereof. In this context, value ranges limited by numerical values are considered inclusive of the cited numerical values in all cases.

Accordingly, a wound treatment article (100, 200, 300, 400) is provided that includes fibers (1.1) that have a high dehydrating capacity; and wherein the fibers are modified natural fibers or synthetic fibers. In one embodiment of the invention, the fibers (1.1) have a water absorption capacity that is less than their dehydrating capacity. In another embodiment of the invention, the fibers (1.1) are extracted from natural materials by chemical processes; and the natural materials are cotton, cellulose or cellulose-containing materials. The fibers (1.1) obtained from cellulose or cellulose-containing materials preferably have a small hemicellulose and resin component; in some embodiments, the fibers (1.1) are mercerized. The wound treatment article can further include at least one wound exudate-absorbing body (2); and/or at least one sheath (3); and/or superabsorbent polymers (2.1); and/or a section having a material selected from the group consisting of soft foam, a modified cellulose, alginates, and a fleece or an air-laid mat or shavings thereof; and/or at least one fluid-impermeable undergarment protection film section; and/or at least one slit (9) or cutout (8). In certain embodiments, the sheath (3) is disposed about a section of the wound exudate-absorbing body (2). In one embodiment, the material is carboxymethyl cellulose. In another embodiment, the fluid-impermeable undergarment protection film section is disposed between the sheath (3) and the wound exudate-absorbing body (2). In yet another embodiment, the fluid-impermeable undergarment protection film section is a film on the sheath (3). In another embodiment, the cutout (8) forms the inner part of a frame (5). In yet another embodiment, the present invention is a kit for acute, emergency, and military medical use or chronic care comprising the wound treatment article (100, 200, 300, 400) as set forth herein in one or more of its embodiments. For example, the kit can be designed for wound compression or for placing around surgically attached organ/tube drainage lines. Lastly, the present invention includes an embodiment relating to a method for wound treatment using the wound treatment article (100, 200, 300, 400) as set forth herein for use as a wound compress, or for cushioning a wound, or for placing around surgically attached organ/tube drainage lines, or for breaking down a biofilm, or for protecting the skin surrounding the wound from maceration, or for treating chronic, acute, and/or bleeding wounds, burn injuries, wounds induced by trauma events, as a wound packing medium, and/or in operative and postoperative care and for military purposes.

### BRIEF DESCRIPTION OF THE DRAWINGS

FIGS. 1a and 1bA show a cross-sectional view of one embodiment of a wound treatment article consisting of plies with mercerized fiber, cellulose, and superabsorbent polymers;

FIG. 2 shows a schematic representation of one embodiment of a wound treatment article constructed in the form of a frame; and

FIG. 3 shows a schematic representation of one embodiment of a wound treatment article constructed in the form of a slotted compress.

### DETAILED DESCRIPTION OF THE INVENTION

The modified natural fibers used for wound treatment articles of the present invention are preferably chemically or physically modified natural fibers such as cotton. The fibers usually comprise cellulose. Cellulose is an unbranched chain polymer consisting of 1-4 β-glycoside-linked glucose molecules with a chain length between one hundred and ten thousand monomers. In general, the chemically modified fibers are preferably fibers with high dehydrating capacity, i.e., good dewatering capability and high water absorption capacity. Accordingly, fibers with an extremely high dewatering capacity (i.e., dewatering capability) and good water absorption capacity as well, i.e., an outstanding ability to absorb moisture quickly and to release it immediately, are particularly preferred.

Of course, the fiber component used may also be a mixture of natural and synthetic fibers. Extracted chemical cellulose manufactured by pulping wood in the acid sulfite process is particularly suitable since the product has a low hemicellulose and resin content, resulting in a very small amount of yellow and correspondingly excellent white content; it is therefore preferred for applications in which hygiene is most important. One example of natural fibers modified in a sulfite process is PLACETATE-F, which is manufactured by Rayonier Performance Fibers and has an extremely high degree of whiteness. Other modified natural fibers that are suitable for use in the wound treatment articles according to the invention include HPZ, HPZ-III and HPZ-XS from wood pulp, and the modified natural fibers known in the form of bleached chemical cellulose by the name SULFATATE-H-J and produced by the same company that manufactures PLACETATE-F.

Generally, the fibers may be microfibers if their individual filaments are finer than 1 dtex, i.e., they have a weight not exceeding 1 gram per 10,000 meters. The feature “as a proportion” as it appears in the claims is intended to indicate that the fraction of fibers is equal to 100% by weight of the wound treatment article, or, in another embodiment, that the fraction of fibers is in a range from 0.1% or about 0.1% to 99% or about 99% by weight of the wound treatment article as a whole.

In one embodiment, the proportion of fibers is in a range of 5% or about 5% to 20% or about 20% by weight of the wound treatment article as a whole; in another embodiment, the proporation of fibers is in a range of 5% or about 5% to 10% or about 10% by weight of the wound treatment article as a whole; such a proportion of fibers is particularly suitable for use in a wound treatment article for treating moist wounds, but could dress wounds of lesser levels of exudation.

In another embodiment, the proportion of fibers according to the invention is in a range of 90% or about 90% to100% or about 92% to about 98% by weight of the wound treatment article as a whole; in yet another embodiment, the proportion of fibers according to the invention is in a range of 92.5% or about 92.5% to 98% or about 98% by weight of the wound treatment article as a whole; such proportions of fibers are particularly suitable for use in a wound treatment article for treating dry wounds with little exudation, but could dress wounds of greater levels of exudation.

In a further embodiment, the proportion of fibers is in a range of 11% or about 11% to 89% or about 89% by weight of the wound treatment article as a whole; in a yet further embodiment, the proportion of fibers is in a range of 20% or about 20% to 75% or about 75% by weight of the wound treatment article as a whole; in another further embodiment, the proportion of fibers is in a range of 35% or about 35% to 65% or about 65% by weight of the wound treatment article as a whole; such proportions of fibers are particularly suitable for use in a wound treatment article for treating wounds that must be kept warm and protected by padding.

However, the fiber proportion may also be interpreted to mean that the fiber is present as a homogenous layer consisting only of fibers and with a proportion by weight of 100%, or that this layer is proportionally mixed with other fibers/substances, in which case the proportion of fibers according to the invention is less than 100% by weight. A fiber proportion of 100% by weight lends itself particularly well to use in a wound treatment article according to the invention that may be placed under one of the wound dressings already known on the market. In particularly preferred manner, a wound treatment article according to the invention is provided that has a cutout in the area of the wound, the dimensions of which correspond to the dimensions of the dressing to be applied, so that the wound treatment article according to the invention is positioned underneath the dressing in the manner of a frame, such that the edges of the wound are effectively kept dry, and the frame provides protection against maceration of the wound edges. The cutout having the same size as the wound dressing also allows unrestricted contact between the dressing and the wound.

Fibers having an enhanced dehydrating capacity according to the interpretation of the present application are already known from hygienic and domestic applications, and to some degree also from textile applications. However, their use in wound treatment articles has not been suggested previously because, until the time of this application, the most frequently used methods for modifying natural fibers were chemical processes such as mercerization or the sulfite process, which were highly likely to render the fibers toxic to cells, and would therefore have a toxic effect particularly upon contact with a wound.

However, the inventor of the present invention has now discovered for the first time that the modified natural fibers have a number of excellent physical properties that are not present in conventional natural fibers, which make them highly suitable for use in wound treatment articles. For example, the modified natural fibers feature good moisture absorbency combined with the ability to conduct moisture away quickly, so that adjacent layers are dried, kept dry, or redried. It was also discovered in the toxicity test that the modified natural fibers do not present any greater toxicity to cells.

It is particularly advantageous that the fibers have the aforementioned properties in both the wetted and unwetted state. The essential feature of the present invention is that it proposes for the first time that the excellent physical properties of chemically modified natural fibers be rendered usable for the treatment of wounds.

Although modified natural fibers have been known for many years—for example, in the form of mercerized fibers, which were known as early as the middle of the 19th century—in addition to their suitability for domestic and hygiene applications, the use of these fibers, or any other chemically modified fibers (for example those fiber materials known as “curled fibers”), or the chemical cellulose produced from cellulose via the sulfite process, for the treatment of wounds has never before been proposed. Among the main reasons for this is that until now the technical community has always thought only in terms of swelling and/or moisture-absorbing materials, such as cellulose, carboxymethyl cellulose, or alginates, for purposes of wound care. Most recently, the use of superabsorbent polymers has been studied intensively, an approach to which the applicant for the present invention also contributed substantial pioneering work.

The use of modified natural fibers with high dehydrating capacity proposed here for the first time runs counter to this mode of thinking, and the specific advantages for purposes of wound care offered by modified natural fibers with high dehydrating capacity can only be discerned if this attitude is overcome—as the applicant for the present invention has done.

The fact that the modified natural fibers have a low moisture absorption capability reduces, for example, the wound edge maceration described above, since the sections that are in contact with the wound edges contain little moisture.

The moisture-conducting properties are particularly advantageous when the modified fibers are arranged in a wound treatment article in combination with a section that absorbs fluids, preferably in such a way that the fibers come into direct contact with the wound, while the fluid-absorbing section is arranged in a second layer. In this way, escaping exudate passes quickly through the fiber section (which functions as a kind of transit layer, in a manner similar to that known from sports undergarments) and is transported to the absorbing section; the fiber-containing section that is in contact with the wound thus remains dry.

Moreover, unlike wound treatment articles with, for example, moisture-absorbing cellulose fibers, a wound treatment article collapses to a greater or lesser degree as the moisture is transported rapidly in the sections containing the modified fibers, thus preserving the structural integrity of the wound treatment article, or it is restored by the quick drying process, which is particularly favorable for padding and heat insulating properties.

Because moisture is transported away rapidly, it is also prevented from diffusing laterally within the wound treatment article, which might otherwise also cause maceration of the healthy skin surrounding the wound. Thus it was determined for the first time that, due to the synergy effects between the absorption element developed specially by the applicant and the modified fibers, it is no longer necessary for the fibers to be oriented perpendicularly to the absorption body, since the absorption body itself is capable of directing the flow of fluids in a targeted fashion. This means that the modified fibers according to the invention are able to prevent lateral diffusion of moisture even more effectively than has been described in the case of microfibers, for example.

The rapid transport of moisture also prevents a damp environment from developing inside the wound treatment article, which would promote the growth of microorganisms, particularly bacteria.

As has already been described with regard to wound treatment articles comprising microfiber layers, the wound treatment article according to the invention is suitable for breaking up biofilms on the wound.

These biofilms consist of a thin layer (film) of mucus in which microorganisms (e.g., bacteria, algae, fungi, protozoa) are embedded. Biofilms are created when microorganisms become established on boundary surfaces. They form mainly in aqueous systems, either on the surface of the water or on the boundary with a solid phase, such as the skin. Biofilms are particularly implicated in chronic and acutely infected wounds because they inhibit metabolic exchange with the environment, obstruct wound cleaning, and threaten wound healing due to their proteolytic properties as well as the endotoxins they excrete.

The inventor has observed that biofilms in wounds usually cannot be combated using antibiotics or disinfectants. Such agents succeed only in killing the bacteria in the topmost layer of the biofilm while the layers below remain unaffected, and in some cases microorganisms in the lower layers may even benefit from the remains of the dead bacteria.

Due to the effect of the modified fibers in demoisturizing or keeping dry the adjacent layers of the biofilm, the biofilm itself is dried out, one of the consequences of which is that the biofilm starts to crack, but more importantly communication between the microorganisms embedded in the biofilm is suppressed. In particular, this also prevents the microorganisms from developing resistance when antibiotic treatment is administered at the same time.

Breaking down the biofilm also helps to improve metabolic exchange between the wound and the environment; general healing promoters, particularly antibacterial, nutritive, or protease inhibiting agents such as are disclosed, for example, in the present applicant's patent application DE102007030931, may be introduced into the wound, thereby promoting wound healing.

In addition, an absorbent element may also be provided that is capable of absorbing the bacteria that become detached from the biofilm or the released components thereof (particularly endotoxins). This topic will be addressed in detail later.

For the patient receiving treatment, the sensation of demoisturizing or keeping dry the layers adjacent to the modified fibers, which may also be the layers of the patient's skin, is an extremely pleasant one since, as described previously, maceration of the intact skin layers is effectively prevented for the first time.

As has already been described with reference to microfibers, the modified fibers and the wound treatment articles made therefrom adhere poorly to the wound surface; combined with their enhanced dehydrating capacity, this results in less painful damage to the wound when bandages are changed, thus enabling bandages to be changed with almost no distress to the patient.

In general, it may also be provided that the modified fibers are present in the wound treatment article according to the invention in the form of cotton wadding, a non-woven fabric, a pile material, terry towelling, flocked towelling, a textile, or a knitted fabric.

The referenced non-woven fabrics may have the form of a non-woven synthetic fleece, for example. Such a fleece may also be made from simple carded fiber wadding. In such an embodiment, the fibers form a fleece having a density between 5.0 g/cm² or about 5 g/cm² and 50.0 g/cm² or about 50 g/cm². In another embodiment, the fibers form a fleece having a density between 6.0 g/cm² or about 6 g/cm² and 20.0 g/cm² or about 20 g/cm².

The referenced piles consist of a microfine fiber material flocked on a carrier material, but they may equally well consist of a matrix textile or knitted fabric into which pile yarns have been incorporated.

It is also conceivable for the modified fibers to be combined with absorbent threads as a monofilament in a composite material, in which case the absorbent threads and the monofilaments are essentially the same length and are fastened in a textile, woven, or knitted backing fabric or in a rigid backing plate by welding or bonding. In this context, the proportion of monofilaments may be from 5% or about 5% to 50% or about 50%, usually between 10% or about 10% and 40% or about 40%, of the total number of absorbent threads and monofilaments. In textile engineering terms, the method that lends itself most readily to production in this context is the double plush method; the monofilaments are worked into a backing fabric together with the absorbent threads as the pile. They should be anchored in the backing fabric by welding, and for this purpose weldable fibers may be included in the material of the backing fabric in the known manner, for example. The monofilaments are fixed securely in the welded fabric. They cannot penetrate and pass through the back of the backing fabric despite the considerable force with which they are braced in the fabric due to their rigidity. The welding also provides a firm, unbroken border around the entire circumference of the monofilaments, so that bending moments are transferred completely to the fabric and the monofilaments cannot be bent by shearing forces with respect to the backing fabric. The backing fabric may be constructed with sufficient rigidity to withstand the bending forces.

The referenced advantages of the modified fibers in conjunction with their use according to the invention in a wound treatment article are realized in any and all of the embodiments described.

In general, the modified natural fibers are conceivable in hydrophobic or hydrophilic form, and usefully employed in the context of the present invention.

In contrast to hydrophilic fibers, hydrophobic fibers do not absorb any fluid or water (i.e., they do not swell), but they are wettable and may therefore store fluid temporarily in a three-dimensional fiber structure. Unlike swelling fibers, however, they release the fluid very quickly again, for example, when an actively absorbent wound dressing, such as the SORBION SACHET, is placed underneath them. (The SORBION SACHET product is available from Sorbion AG of Senden, Germany.)

The fibers have moisture-conducting properties in this embodiment as well and they particularly prevent the establishment of a microclimate favorable to the growth of bacteria.

A mixture of hydrophilic and hydrophobic fibers may be provided particularly in this context. In this way, for example, dedicated drainage zones having hydrophilic microfibers may be formed in the wound treatment article, whereas surrounding zones having hydrophobic fibers may channel the exudate towards these drainage zones.

In this case, it may also be provided for the modified natural fibers to be woven or twisted together to form yarns or multifilaments, for example, before they are processed to produce a nap or cloth.

In embodiments that include such a nap or cloth, the wound treatment article is typically used as a wound dressing. The referenced wound dressing may then be flat, for example, and be round, elliptical, rectangular, or polygonal in shape. However, the wound treatment article of such embodiments may additionally or alternatively be a wound cleaning cloth, a swab, a pad, a wound packing item, or the like. These items may have absorbent or non-absorbent properties. The wound treatment article according to the invention having a proportion of chemically or physically modified fibers may also be capable of being cut and may have the form of a tube bandage.

In yet other embodiments, the wound treatment article includes at least one body designed to absorb wound exudates and a sheath arranged to surround at least sections of this body or bodies.

In this context, the body for absorbing wound exudates can include a material in the form of a mat. One useful embodiment of the mat is one formed from an air-laid material including superabsorbent polymers and/or a loose filling of suberabsorbent polymers. This air-laid mat can have an essentially flat material section of absorptive material, consisting for example of an absorbent fleece with superabsorbent polymers distributed therein.

This exudate-absorbing body may be equivalent to the absorbent insert contained in the present applicant's wound dressing as disclosed, for example, in WO03094813, WO2007051599 and WO0152780 and is marketed under the aforementioned trade name SORBION SACHET. The disclosures of the documents cited here are incorporated in their respective entireties herein.

In a different configuration, the exudate-absorbing body may also form a core that contains—possibly flake-like—fibers or yarns of superabsorbent polymers and superabsorbent polymer granules, the granules being bonded or fused to the fibers or yarns on multiple levels, and the granules being distributed over more than 50% or more than about 50% of the total height of at least one section of the core, and the granules and fibers being present together in certain zones. The proportion by weight of superabsorbent polymers may be in the range of 10% or about 10% to 25% or about 25% by weight for use as a wound compress. In another embodiment, the proportion by weight of superabsorbent polymers may be in the range of 15% or about 15% to 20% or about 20% by weight for use as a wound compress.

In another design, the exudate-absorbing body may also contain at least one flat ply having fibers or yarns of superabsorbent polymers, to which superabsorbent polymers in granule form are bonded. In a related embodiment, the exudates-absorbing body has a body construction of at least three layers, wherein two covering layers surround a layer having superabsorbent polymers.

In this case, there are no mixtures of fibers and superabsorbent polymers in the plane; only fixed juxtapositions of the two materials. In one embodiment, if multiple plies are provided, they may also be physically compressed against each other by rolling, pressing, calendering or similar methods. The body may also be furnished with repeating patterns or markings, for example, a diamond pattern, a stamped pattern, a company logo, or similar.

A sheath may be arranged around the core such that it overlaps in certain zones and, for example, either covers an adhesive seam or is part of such a seam.

It is also possible for a section of a hydrophobic and/or water-repellent or water-impermeable material to be provided inside the sheath in order to prevent moisture from soaking through or to protect undergarments.

In yet another embodiment, it is provided that the sheath has the modified natural fibers, which are preferably prepared in the form of a woven or knitted material, a non-woven material, or fleece.

The present invention using the indicated sheath provides an effective way to ensure the direct contact between the fibers and the wound that results in the positive effects described above. Equally, the present invention provides for a section including the modified natural fibers to be arranged inside the sheath. The fiber section located inside the sheath then functions exceptionally well in redrying the sheath.

For these reasons, the referenced section is arranged on the side of the wound treatment article facing the wound, and may, for example, consist of a three-dimensional nonwoven web.

Since the modified natural fibers or synthetic fibers are equally capable of being woven or knitted into fabrics, such a section may, for example, consist of a three-dimensional knitted or woven material. Not only does a knitted (or woven) spacer fabric of this type have excellent cushioning properties, it also has good breathability and moisture-conducting properties, thereby ensuring that the wound is allowed to dry even under conditions in which heat and/or sweat are generated to a certain degree, without impairing the level of moisture necessary to promote wound healing.

An embodiment of such woven or knitted fabrics, or even tufted materials made from chemically and/or physically modified fibers, may include a juxtaposition or blending of two different materials, for example, with one of the materials being hydrophobic and the other hydrophilic.

Thus, for example, it is conceivable to create a discontinuity in the ply forming the modified natural fibers by means of ribs or wicks of carboxymethyl cellulose or polyurethane, which transport the wound fluid away in a targeted manner similar to the effect of a chimney. This is particularly true when no absorption body such as was developed by the applicant has been used as an underlay.

In another embodiment of the present invention, the modified natural fibers consist of biodegradable materials that may be coated with a biodegradable magnesium alloy, as is known from surgical suture materials, thereby supporting biocompatibility as well as the body's own healing and defense mechanisms in the wound healing process.

In particular, the sheath may also have a pattern of materials containing various modified natural fibers. For example, the sheath may include a striped or diamond pattern, in which individual sections conduct moisture away more effectively than other sections.

In another embodiment, it is further provided that the wound treatment article also includes superabsorbent polymers.

Superabsorbent polymers (SAP) are plastics that are capable of absorbing many times their own weight—as much as 1000 times—in fluids. In chemical terms, these substances are commonly copolymers of acrylic acid (propene acid, C₃H₄O₂) with sodium acrylate (sodium salt of acrylic acid, NaC₃H₃O₂), the relative proportions of the two polymers being variable. In addition, a core cross linker (CXL) is added to the monomer solution to bond the long-chain polymer molecules to each other at certain points via chemical bridges (“crosslinking”). These bridges render the polymer insoluble in water. When water or aqueous salt solutions penetrate the polymer particle, it swells up and strengthens this network at the molecular level, with the result that the water can no longer escape unassisted.

The use of SAPs in wound dressings to trap exudate is known from WO0152780 and WO03094813 of the present applicant, and the disclosures thereof are incorporated in their respective entireties into the present application. The term “exudate” refers to a wound fluid derived from blood plasma due to the inflammatory processes of wound edema. Just as blood is responsible for transporting nutrients and other transmitters, and thus for nourishing various parts of the body, exudate serves in a very similar way to supply the wound bed and the healing processes that are taking place there. In order to be able to perform these many functions properly, it contains a wide range of components, resulting in a specific weight slightly greater than that of water. In this respect, it also differs from transudate, which is caused by non-inflammatory processes and has a considerably lower specific weight and a lower cell and protein content. In addition to providing nutrients for the fibroblasts and epithelial cells, exudate coordinates the various wound healing processes both temporally and spatially with its high content of growth factors and cytokines. These consist mainly of thrombocytes, keratinocytes, macrophages, and fibroblasts. They affect the motility, migration, and proliferation of the various cells involved in wound healing. For example, they stimulate migration of cells into the base of the wound as well as supplying the newly formed granulation tissue by angiogenesis. The exudate even assists in cleaning the wound. It contains various serine, cysteine, and aspartate proteases as well as matrix metalloproteinases, whose activity is very tightly controlled and includes breaking down existing and newly formed collagen in the wound.

The main components of physiological exudate include salts, glucose, cytokines and growth factors, plasma proteins, proteases (particularly matrix-metalloproteases), granulocytes, and macrophages.

If the wound healing process does not show significant progress in keeping with the various phases of wound healing within a few weeks, the wound is termed chronic. At the same time, however, exudative phases lasting longer than three days are considered to be a complication, and are described as pathological exudation, which can contribute to the chronification of the wound. The underlying causes are usually complex and may well be systemic in nature. However, given the important role of the exudate in wound healing as described previously, it is not surprising that complications in healing are reflected in significantly altered composition and effect of the exudate.

Among other consequences, the concentrations of individual components of the exudate undergo a shift in the case of chronic wounds, and the beneficial effect of substance, which supports healing in normal situations, is lost. In particular, the content of inflammatory cytokines and proteases is significantly elevated in pathological exudate. Conversely, the growth factor content is lowered. A particularly serious change occurs with regard to the activity of the matrix-metalloproteinases mentioned previously. In addition to preparing the wound bed, these substances are also involved in the subsequent conversion of granulation tissue to scar tissue. These enzymes are normally present in the form of an inactive pre-enzyme, and their activation is regulated by corresponding inhibitors (tissue inhibitors of metalloproteases, TIMPs) which also help with cell growth at the same time. In chronic exudate, protease activity seems to be increased as a result of the disturbances in this regulatory system, and this may contribute to regression in wound healing. In pathological exudate, the balance of the components necessary to promote wound healing has been lost. This leads to a series of complications, which contribute to the deterioration and chronification of the wound.

In conjunction with the dehydrating properties of the modified natural fibers or synthetic fibers of the wound treatment article according to the invention, which are able to dry out and thus break up biofilms present in the wound, the component containing SAP described above assumes a particular significance. Thus, the SAP fraction provided serves to absorb the fragments and remains of the biofilm. With the destruction of the biofilm, any endotoxins and bacterial pathogens (particularly bacterial hemolysin and leukocidin), which can provoke inflammations, allergies, shocks (particularly anaphylactic shocks and/or toxic shock syndrome), and fevers (Herxheimer reaction) in the patient, are released. These endotoxins and pathogens are absorbed by the SAP fraction, and the consequences described above may be avoided.

These circumstances are described thoroughly in DE102007054127, which is held by the present applicant, the disclosure of which is incorporated herein by reference; in particular, it is demonstrated therein that SAPs are capable of binding bacteria and bacterial endotoxins.

The superabsorbent polymers are preferably polymers in powder or granulate form.

In this administration form, superabsorbent polymers offer a large number of advantages, and are known, for example, from WO0152780 and WO03094813, which are held by the present applicant.

The superabsorbent polymers usefully employed in the context of one embodiment of the present invention are polymers in the form of fibers, yarns, cotton wadding, fleeces, or woven fabrics. In this context, reference is made to DE102007049429, held by the present applicant, the disclosure of which is hereby incorporated herein by reference.

The proposed use of superabsorbent polymers in the form of fibers or yarns has a number of advantages over particulate superabsorbent polymers:
i) Such fibers or yarns have a wicking effect. They are thus able to absorb and bind a fluid very much more quickly upon contact with it than can superabsorbent polymers in granulate form.
ii) Moreover, they are able to direct and channel the fluid flows. This may help to prevent wound maceration, for example.
iii) Unlike particulate superabsorbent polymers, they may be processed to form fleeces, woven fabrics, or the like. Thus, for example, the sheath of a wound dressing may be manufactured from these fibers or yarns, allowing the superabsorbent properties to be introduced much closer to the base of the wound.
iv) The danger that superabsorbent materials may be left behind in the wound is much lower for a woven fabric of superabsorbent yarns than for particulate superabsorbent polymers. This also helps to ensure that the superabsorbent properties may be applied much closer to the base of the wound.
v) Since a separate carrier material may be omitted in many cases, the fraction of superabsorbent materials in a hygienic or treatment article, particularly a wound care article, may be increased considerably; in extreme cases, it may even account for a portion of 100% by weight.
vi) These fibers or yarns and the products produced therefrom are much softer and less abrasive than the corresponding particulate superabsorbent polymers.
vii) These fibers or yarns may be processed to create a structure without the need to use an adhesive or welding process, as is necessary with particulate superabsorbent polymers. This has significant advantages both with regard to the purity of the product and with regard to pharmacology and possible allergenicity.
viii) Unlike the particulate superabsorbent polymers, the dimensions of these fibers or yarns maybe controlled and checked much more precisely, preventing the production of dusts, such as are often generated when particulate superabsorbent polymers are used, and also substantially improving product quality (homogeneity and reproducibility).
ix) Since much less dust is generated, it may also be possible to omit a separate sheath.
x) In the air-laid materials known from the related art, which contain particulate superabsorbent polymers, these yarns or fibers may replace the carrier fibers of the air-laid materials, thereby increasing the proportion of superabsorbent materials in a hygienic or treatment article, particularly a wound care article, and thus also the total absorption capacity.

Any two- or three-dimensional arrangement of fibers or yarns is conceivable here. For example, the fibers or yarns may be aligned or unaligned (tangled), organized in multiple plies, or in any other arrangement.

Superabsorbent fibers made from polyacrylates are marketed and sold commercially, for example, by Technical Absorbents (Grimsby, UK) under the trade name OASIS SUPER ABSORBENT FIBER or OASIS SAF. Like all superabsorbent polyacrylates, they have an extremely high capacity for absorbing fluids. These fibers may be produced in the form of a fleece, an air-laid material, a woven fabric, and/or a non-woven fabric, for example. Such fibers are known, for example, from DE 69807337.

However, these fibers made from superabsorbent material are usually very fragile, because the polyacrylate material used is very brittle. For this reason, the fibers cannot exceed a certain length and therefore cannot be easily made into a yarn.

In contrast, WO0106047 describes yarns that contain superabsorbent polymers. These yarns are based on a special manufacturing process in which the superabsorbent fibers described above are mixed with reinforcing fibers made from a stronger material and then spun to form a yarn. These reinforcing fibers may be, for example, fibers made from polyester, polypropylene, nylon, cotton, viscose, or a similar material. The yarn thus produced may be processed to yield a woven fabric, a mesh material, a knitted fabric, and/or a non-woven fabric. The fibers may also be processed with elastic fibers, for example, fibers made of elastane.

Until now, however, the use described for such yarns has been for cables, particularly submarine cables, for example in EP1072698, which was submitted by the same applicant as WO0106047 discussed above.

In another embodiment of the present invention, the suberabsorbent polymers available in the form of fibers are present at least in part in the form of a cotton wadding, a fleece, an air-laid material, and/or a non-woven fabric.

To this end, fibers are used having an average length of 5 mm or about 5 mm to 50 mm or about 50 mm. Production is carried out according to known methods, such as carding or the air-laid process. Here, the superabsorbent fibers may be the sole constituent of the material in question. However, in a related embodiment, the hygienic or treatment article also includes a proportion of reinforcing fibers that guarantee the integrity of the wound dressing even when it is damp, such fibers being selected from the group including:
a) Cellulose fibers,
b) Viscose fibers,
c) Alginate fibers, and/or
d) Polyester, polyolefin, polyurethane, polyvinyl alcohol, or polyamide fibers, and/or mixtures or copolymers thereof.

In this way, the absorption properties of the material concerned as well as other properties such as ductility, tear strength, behavior when saturated, and the like may be controlled in a precise manner.

For example, in another embodiment of the present invention, a woven fabric is provided in which, for example, the warp threads are made from superabsorbent yarns while the weft yarns are made from other yarns, including, for example, reinforcing yarns in accordance with the above list. Such a woven fabric has targeted hydroactive properties, i.e., it absorbs a fluid in one direction and transports it.

At least some of the fibers may preferably be produced in the form of a fleece, a woven fabric, a knitted fabric, an air-laid material, and/or a non-woven fabric.

It may further be provided that the modified natural fibers, or at least sections thereof, are present in a composite with other materials, particularly other fibers.

These other materials may be, for example, cotton wadding, fluff pulp, medical cotton, spun rayon, fluff, regenerated cellulose, shavings, rayon, viscose, modal and/or Tencel.

In the remainder of this document, the term “shavings” will refer to particles obtained from a cellulose-containing material by shredding, punching or cutting. Such shavings may be produced for example from an air-laid material, which may contain superabsorbent polymers and is described in the latter form in WO03094813, held by the filer of the present invention.

In the following text, the term “spun rayon” will be used to refer to a material consisting of pulp and with characteristics that lie between cotton wadding and wool.

In the following text, the term “fluff” will be used to refer to flaky pulp particles such as are used in diaper technology.

Regenerated cellulose is the term used generally to refer to fibers such as viscose, LYOCELL (manufactured by Lenzing AG of Lenzing, Austria), modal, rayon, or cupro fabric.

“Fluff pulp” is a standardized wood product that is produced, for example, from cellulose material supplied as panels or webs, also known as wood pulp cardboards, and which is usually shredded in hammer mills or chipped and roved with scraper devices before use until a product resembling cotton wadding is obtained from cellulose fibers. The material thus obtained is preferably bleached without the use of chlorine.

Such a mixture may have a wicking effect and thus improve the moisture conducting-properties of the wound treatment article according to the invention.

Fibers of superabsorbent polymers and/or fibers made from modified cellulose, particularly carboxymethyl cellulose (CMC), may be considered in this instance as well. However, a composite of this kind would lose the positive properties of the modified natural fibers (rapid moisture transport) described above.

A mixture of CMC or fibers of superabsorbent polymers and the modified natural fibers is partly transformed into a gel when absorbing moisture, but nonetheless retains its structural integrity. Reference is hereby made to the present applicant's patent applications DE102007049429 and DE102007049430, the respective disclosures of which are hereby incorporated by reference.

Overall, the moisture-conducting and moisture-binding properties of the wound treatment article according to the invention may be controlled in an extremely precise manner by deliberate adjustment of the mixture ratios between the modified natural fibers and other fibers, or between the hydrophilic and the hydrophobic fiber types. In this context, a gradient may be provided in the fiber mixture ratio throughout the depth of the wound treatment article or a section thereof.

In addition, the sheath material preferably includes at least one portion that is hydrophobic and/or does not adhere to the wound.

Such a material effectively prevents the dressing from adhering to the wound, thus reducing distress when bandages are changed. Said material is particularly preferably a mercerized cotton, though regenerated cellulose such as viscose, Lyocell, modal, rayon or cupro fabric, polyamide (nylon), polyethylene, polypropylene, PTFE (Teflon), siliconized fibers, or similar materials may be considered as well. In particular, the fibers may also be straight, wavy, curled, and/or mercerized, fibers from natural substances being particularly preferred.

In another preferred embodiment, the sheath may include a layer containing superabsorbent polymers in fiber form. Such a sheath is described, for example, in DE102007036758, which is held by the present applicant, and the contents of which are included in their entirety by reference.

In this context, it is particularly preferable for the material to have a three-dimensional pore structure, such as is described in WO2007118652, which is held by the present applicant, and which is hereby incorporated herein in its entirety by reference.

In some embodiments of the present invention, this sheath may consist of or comprise a non-woven material of polypropylene or polyethylene having a weight per unit area in the range from greater than or equal to 10 g/m² or about 10 g/m² to less than or equal to 100 g/m² or about 100 g/m²; in another embodiment, the non-woven material included in the sheath is included in a range from greater than or equal to 20 g/m² or about 20 g/m² to less than or equal to 40 g/m² or about 40 g/m².

It is also possible to include in an embodiment of the present invention a section of a hydrophobic and/or water-repellent or water-impermeable material inside the sheath to prevent moisture from soaking through or protect undergarments.

In such an embodiment, the superabsorbent polymers used are immobilized inside the wound dressing. This is important in order to prevent the polymers from leaking out of the dressing and into the wound.

There are a number of ways to immobilize the polymers. For example, without intending limitation as to method for such immobilization of the polymers, one or more of the following procedures for such immobilization may be employed:
a) the superabsorbent polymers are bonded to fibers of the wound dressing, for example, using a hot melt adhesive;
b) the superabsorbent polymers are pressed together with fibers of the wound dressing;
c) the superabsorbent polymers are embedded securely in a three-dimensional fiber matrix;
d) the portion of the dressing containing the superabsorbent polymers is enclosed in a sheath that is impermeable to the polymer, for example, by selection of a suitable pore size or because it is non-porous (this may be the outer sheath of the wound dressing, or it may also be an inner sheath arranged inside the outer sheath);
e) the superabsorbent polymers are present in the fiber form, the fibers being present in the form of a fiber composite and may even be joined in composite with the pulp fibers of the dressing.

In one embodiment, the sheath material contains no intentionally introduced pores; any pores present in the material are created by the mesh width in the woven fabric or the distance between fibers of the fleece. In another embodiment, the material of the sheath is deliberately furnished with pores; the pore size is determined on the basis of the size of the superabsorbent particles and may have values in a range from less than or equal to 50 μm or about 50 μm to less than or equal to 5000 μm or about 5000 μm; in yet another embodiment, the range is less than or equal to 100 μm or about 100 μm to less than or equal to 3000 μm or about 3000 μm.

In another embodiment, the wound dressing of the present invention also has
a) a section having soft foam
b) a section having modified cellulose, in particular carboxymethyl cellulose, and/or
c) a section having alginates, and/or
d) a section having a fleece and/or an air-laid mat, or shavings thereof

The term “Airlaid” refers to a special fleece made from pulp and polyolefin fibers, in which superabsorbent polymers may be embedded. This may be, for example, the air-laid mat contained in the aforementioned SORBION SACHET product, which is described in WO03094813, held by the present applicant.

In this context, a proportion of pulp may be positioned above or below the air-laid mat, this portion being, for example, a portion of cotton wadding, fluff pulp, medical cotton, spun rayon, fluff, regenerated cellulose, shavings, rayon, viscose, MODAL and/or TENCEL, and containing a portion of superabsorbent polymers whether or not the air-laid matalso includes a portion of such superabsorbent materials.

The term “fleece” refers to a textile material consisting of individual fibers which, unlike woven or knit fabrics, is not manufactured from yarns. Fleeces typically retain their structural integrity by adhesion of the individual fibers to each other. They are also called “nonwovens” and are produced, for example, by carding the fibers.

The referenced foam may, for example, be a material selected from the group including thermoplastic soft foams such as polyurethane foam, polyamide foam or polyether foam, silicone foam, as well as cellulose foam or natural sponge. In one embodiment of the present invention, this foam provides structurally stable properties.

Natural sponges, for example, from the class of demosponges (Demospongiae), demonstrate a capacity to absorb fluids similar to that of technical foams. They also have growth-inhibiting properties with regard to microorganisms in order to be able to protect themselves from colonization by sessile organisms. These properties may also be useful in the context of wound care for preventing bacterial growth in the wound dressing and/or in the wound. These sponges also have properties that inhibit the growth of fungi and protozoa. Moreover, sponges of this kind are able to absorb fluids and are ideally suited to absorbing exudates.

This natural sponge may be placed over the wound in thin strips which have been created by thermal cutting, for example.

The soft foam material may optionally be constructed in multiple plies, the individual plies having thicknesses in some embodiments of the present invention between 0.5 mm or about 0.5 mm and 10 mm or about 10 mm; and in other embodiments, between 4 mm or about 4 mm and 6 mm or about 6 mm.

The soft foam material may have an open cell or closed cell structure. Particularly in the latter case, it may be provided that the material includes the cuts and/or perforations that accelerate the flow of the fluid into and through the soft foam material. The soft foam material may also be in the form of an integral foam.

Alginates are obtained from brown algae and woven into a fibrous fleece. In chemical terms, they are polysaccharides, specifically calcium and/or sodium salts of alginic acids. Alginates are able to hold up to 20 times their own weight in fluid, and in this case the wound exudate is deposited in the cavities thereof. The Ca²⁺ ions in the alginate lattice are exchanged with the Na⁺ ions from the exudate until the Na ion saturation level is reached in the alginate. At the same time, the wound dressing swells and the alginate fibers swell up and are transformed into a gel.

Carboxymethyl cellulose exists primarily in the form of sodium carboxymethyl cellulose and is available commercially under the name HYDROFASER. In hygienic and wound treatment products, the fibers are converted into a flat matrix. As they absorb fluid from the wound exudate, the fibers are gradually transformed into a gel cushion that retains the fluid and does not release it again. The structure of the fibers is such that the wound exudate is only absorbed vertically. This means that, as long as they have sufficient capacity, the exudate will not flow over the edges of the wound. This effectively prevents any maceration of the wound edges.

All of the materials and sections listed in items a-d above may be organized in a sandwich arrangement above and/or below the body that is designed to absorb the wound exudate and contains a component of pulp. However, it may equally well be provided that the former are integrated into the exudate-absorbing bodies in the manner of an insert, for example, as a strip, a round section or a cruciform section. In this case, the exudate-absorbing body is furnished with a correspondingly shaped window or at least a cutout on one side. Conversely, it may be provided that one of the sections listed in items a-d itself forms a window or at least a one-sided cutout in which the exudate-absorbing body including a pulp component is arranged.

It is also preferably provided that at least one fluid-impermeable film section for protecting undergarments is arranged between the sheath and the exudate-absorbing body. This film section may preferably be positioned such that it only covers the middle zone of the wound treatment article.

Additionally, in another embodiment, a kit for acute, emergency, or military and chronic care is provided, including optionally a wound treatment article as also described herein.

Such a kit can be provided for compressing a wound and/or surrounding surgically inserted drainage tubes.

According to one embodiment of the invention, it is further provided to use a wound treatment article as a wound compress, for cushioning a wound, to reposition surgically inserted drainage tubes, to destroy a biofilm, and/or protect the borders of the wound from maceration.

According to another embodiment of the invention, it is also provided to use a wound treatment article or a kit to treat chronic, acute, and/or bleeding wounds, burn injuries, injuries caused by trauma, as a wound filler, and/or in operative and postoperative care, and for military purposes.

At this point, it should also be noted that chemically and/or physically modified fibers of the present invention can be employed for use as wound filling media. Chronic wounds are often accompanied by considerable loss of tissue, which is preferably filled out, not only so that the exudate escaping onto the wound base can be conveyed away, for example, into an absorbent cushion placed above it (wicking effect), but also to provide a soft padding for the wound, which is extremely sensitive to pressure and pain. The second purpose is particularly important for psychological reasons, because such a loss of tissue is a significant source of concern to the patient. Such a wound filling agent may thus be thought of as a temporary prosthesis to provide the patient with the impression that the tissue loss has been corrected—at least temporarily. The chemically and/or physically modified fibers proposed according to the invention, which are preferably used for this purpose in the form of a cotton wadding or nonwoven wadding fleece, are well suited for this purpose.

In addition, the invention also provides a method for treating wounds including the use of a kit or a wound treatment article.

### Variants

The optionally provided sheath described in the context of some embodiments of the present invention preferably consists of at least in part of a hydrophobic material, for example, polypropylene, polyethylene, or a natural material having hydrophobic properties such as cotton. The hydrophobic properties of the sheath prevent adhesion with the surface of the wound and serve to enable the wound exudate to penetrate the inside of the sheath more quickly. In a preferred embodiment, the sheath consists of a perforated film material, for example, polypropylene or polyethylene, and is also furnished with an underlay of a film material such as polypropylene or polyethylene. In a particularly preferred embodiment, the film material used as the underlay is laminated below the perforated film material, and the film material of the underlay preferably has a weight per unit area of 1.5 g/m² or about 1.5 g/m² in some embodiments, in other embodiments 2 g/m² or about 2 g/m², and in yet other embodiments, 3 g/m² or about 3 g/m².

In other embodiments, the sheath is made of a different plastic, particularly a polyurethane or polyethylene film, or a film of synthetic spider silk.

The sheath material may be structured such that the sheath has a rough inner surface and a smooth outer surface. The rough inner surface of the sheath is preferably formed by funnel-shaped perforations, each of which tapers toward the inner surface and terminates in a free opening edge (“projection”). This rough inner surface prevents the contents of the sheath from shifting, so that it is not necessary to affix it with adhesion points. Accordingly, the smooth outer surface of the sheath material may be formed by convex material sections extending between the perforations. Such a sheath material may be termed “three-dimensional” in contrast to one that is flat on both sides, and is known, for example, from DE102006017194, held by the present applicant, the entire disclosure of which is hereby incorporated herein by reference.

In addition, the sheath of a wound dressing according to one embodiment of the invention to have an adhesive coating at least on sections thereof, preferably but not necessarily on the side facing away from the wound, which may be used to secure it in the area of the wound, for example, using a bandage.

It may also be provided that the sheath has a zone that projects over the actual wound and that is provided with an adhesive strip for securing it.

Additionally, in some embodiments, a material section may also be provided on the site of the wound dressing facing away from the wound, said section extending beyond the limit of the sheath itself and having an adhesive coating facing the skin at least in its border areas, for example, in the form of adhesive strips or patches (known as an “island dressing”).

This flat material section may particularly have semiocclusive or semipermeable properties, i.e., it may be permeable to moisture but not to bacteria.

Here, the permeability for water vapor is preferably in the range of greater than or equal to 500 g or about 500 g to less than or equal to 16000 g m⁻² h⁻¹ or about 16000 g m⁻² h⁻¹.

Preferred adhesive materials for the purposes described above are physiologically acceptable agents such as hydrocolloid adhesives or medically harmless adhesives, such as solvent-free, biocompatible silicone adhesives or polyacrylate adhesives, which have good resistance to all common sterilization procedures.

Unlike other plastics, such as polypropylene, polytetrafluoroethylene (Teflon), and silicone, polyurethane foam lends itself well to adhesion.

In a special embodiment, it is provided that the absorbing body is arranged asymmetrically inside the sheath, i.e., predominately on one side.

Such a wound treatment article may particularly be used in wound cavities where space is limited. In this case, the section of the wound dressing in which the absorbing body is located remains partly outside the wound cavity. Exudates that collect in such a wound cavity are effectively taken up by capillary action, which promotes wound healing as well.

Furthermore, in some embodiments, the wound dressing has a lateral incision or a wedge-shaped cutout such that the edges of the incision or cutout may be positioned to overlap each other.

Furthermore, in other embodiments, the wound dressing includes a section that fills out the wound. This section may be designed such that, for example, it swells up upon contact with exudate and spreads to occupy the wound as far as the wound base.

The wound dressing may thus be formed in three dimensions, creating a concave shape so that it may be conformed for application to a joint, an extremity, or a curved body part, for example. The overlapping borders of the incision or cutout may then be secured, for example, using Velcro fasteners, snap fasteners, adhesive strips, or other suitable fixing means.

In another embodiment, it is provided that the wound dressing has a three-dimensional conformation such that it is adapted for application to a joint, an extremity, or a curved body part.

Thus, the wound dressing may have a concave shape, for example, so that it may be adapted for application to a patient's heel or elbow. The absorbent body, which is located inside the wound dressing, may be designed in a removable fashion. This design is particularly advantageous if the absorbent body has been moistened previously or is moistened by escaping exudate, since a gel is formed by the superabsorbent polymers that has a cushioning effect, thus enabling the body part in question to be repositioned painlessly. In addition, moistening also enables the wound dressing to be adapted even more optimally to the existing anatomical conditions.

In certain embodiments of the invention, the wound dressing is present at least partially in a rolled form. In this case, it may be provided that the originally flat wound dressing is rolled up and possibly secured in its rolled form, for example, by stitching, bonding, or welding. Such a wound dressing is particularly suitable for use as a packing medium in wound cavities; in particular, it functions as a wick for the exudate to be absorbed.

In this context, the wound dressing additionally has at least one nutritive agent, at least one disinfecting or decontaminating agent, and/or at least one protease-inhibiting agent and/or active ingredient complex.

The disinfecting agent and/or active ingredient complex may be, for example, a compound of at least one vitamin or vitamin derivative, a metal ion, and a detergent. It may also be a BUS (bacteriocin-like inhibitory substance), an antimicrobial peptide, an antibiotic, a silver preparation, or coated magnetic particles. In one embodiment, a quaternary ammonium compound is the active ingredient included in the product of the present invention.

The agent and/or active ingredient complex having a nutritive function may be a compound containing at least the components of an enteral and/or parenteral dietetic product. It may also be at least one active element selected from the group including insulin, recombinant insulin, proinsulin, an insulin-like growth factor (IGF), an insulin mimetic, and/or a diabetic-specific energy carrier that is not based on glucose or sucrose.

The agent and/or active ingredient complex with nutritive function may also be a glycolytic enzyme, for example, a hexokinase that converts glucose into glucose-6-phosphate, thereby initiating glycolysis and lowering the glucose level, which is frequently elevated in diabetic patients.

The protease-inhibiting agent and/or active ingredient complex may be at least one active element selected from the group including protease inhibitors, superabsorbent polymers, antibodies for matrix metalloproteinases (MMP, particularly for MMP 2, 7 and 9), chelators for divalent cations (particularly for Ca²⁺), collagen, coated magnetic particles, acids, buffers, non-pathogenic acid-producing microorganisms, probiotics, and/or symbiotics.

Here, a carrier substance may particularly be provided in the wound dressing in order to bind divalent cations (particularly Ca²⁺).

Further context and background information regarding agents and/or active ingredient complexes with nutritive, disinfectant or decontaminating, and/or protease-inhibiting functions are provided in DE102007030931, held by the present applicant, the content of which is included in the present application in its entirety by reference. Other agents and/or active ingredient complexes with nutritive, disinfectant or decontaminating, and/or protease-inhibiting functions are also described in DE102007030931, which is hereby incorporated herein by reference in its entirety.

The wound dressing may also include a preparation containing phages and/or components thereof. Such a wound dressing is described in DE102007054127, held by the present applicant, the content of which is included in its entirety by reference.

It may further be provided that the wound dressing contains necrolytic and/or fibrinolytic enzymes. It may also contain angiogenesis- or epidermogenesis-promoting growth factors (particularly from the VEGF and EGF groups). The wound dressing may also contain attractants for macrophages, which engage in phagocytosis of phages and bacterial remains (particularly endotoxins) that are released in the course of phage therapy.

It is further preferably provided that at least one section of the wall of the wound dressing sheath includes a reservoir for at least one active ingredient and/or active ingredient complex having a nutritive, disinfecting or decontaminating, protease-inhibiting, hemostatic and/or wound-healing function.

The referenced reservoir may consist for example of a pouch incorporated into the sheath wall. The reservoir may also consist of a section of the sheath wall that is impregnates with the agent and/or active ingredient complex, or the agent may be impressed into the section.

Substances may be added to the wound dressing to increase the osmotic pressure. Such substances include, for example, osmodiuretics such as mannitol.

Mineral ion exchangers such as zeolites, bentonies, or montmorillonites may also be used as a component of the wound dressing, particularly of its mat. Among their other properties, zeolites are able to absorb toxins such as heavy metals. In addition, they have a hemostatic effect.

The article may also contain activated charcoal, which is preferably dispersed in a thin layer, for example, in a fleece layer, on the side facing away from the wound, and particularly serves to absorb unpleasant odors emanating from the wound site.

The wound dressing may also include a preparation containing phages and/or components thereof. Such a wound dressing is described in DE102007054127, held by the present applicant, the content of which is included in its entirety by reference.

The wound dressing according to the invention may also be incorporated into a wound treatment system to drain wounds using negative pressure. Such systems are disclosed, for example, in DE202004017052, WO2006048246 and DE202004018245, held by the present applicant, the contents of which are to be considered appurtenant to the present invention.

The first document cited discloses a device for treating wounds using negative pressure comprising a gas-impermeable wound covering element which, when applied to the patient's body, forms a durable space between the wound and the wound covering element, and at least one connection point that communicates with the space and via which the air in the space may be evacuated, wherein the said wound covering element covers at least one extensive wound dressing underlay for absorbing the wound exudate, the volume of which underlay increases in the course of the absorption process, so that the absorbed wound exudates remain inside the wound dressing and therefore below the wound covering element until the wound dressing is removed from the patient's body, said wound dressing being at least one ply of a textile section enriched with superabsorbent materials and being enclosed in a liquid-permeable sheath, and the ply having an area on its flat side that is 3% to 90% smaller on its flat side than that of the sheath, so that the wound dressing is able to approximate a circular shape as it approaches its maximum absorption capacity.

The second document cited discloses a multicomponent bandage for treating wounds in a human or animal body using negative pressure and having: a wound covering element for attachment to the skin or mucous membrane surface, at least one connection point that communicates with the wound space and via which substances in the wound space may be evacuated, said space containing superabsorbent polymers, with the absorbed wound exudates remaining bonded to the polymers in the wound space until they are removed from the wound space, and with the binding properties of the polymers promoting mutual synergies with the subatmospheric pressures.

The third document cited discloses a drainage device for wound treatment using negative pressure, including a gas-impermeable wound covering element made from a film-like material which, when applied to the patient's body, is adhesively affixed to the skin surface around the wound site and forms a durable, sealed space between the wound and the wound covering element, at least one drainage tube that may be inserted into the space and via which the substances in the space may be evacuated, and at least one wound dressing arranged inside the space to absorb wound exudates, including at least one ply of a textile section enriched with superabsorbent materials, said dressing being enclosed in a liquid-permeable sheath, wherein the absorbed wound exudates remain inside the wound dressing and thus below the wound covering element until the wound dressing is removed from the patient's body, and wherein the wound covering element has a sealable, gas-impermeable treatment opening, through which the wound dressing may be placed in the space and removed from the space.

Unlike the embodiments shown in the cited disclosures, it is also possible here for an absorption body containing superabsorbent polymers not to be located in the vicinity of the vacuum device. In this case, it is more important that the microfibers—or a fabric, spacer fabric, knitted fabric, or fleece fabric containing microfibers—be provided for padding, cushioning the underside of the device shell, thus ensuring lasting cushioning and permeability for fluids. The structurally stable, non-swelling properties of the microfibers are particularly advantageous here since they continue to exhibit sufficiently high restoring forces even when a partial vacuum usually in the range of 80-250 mmHg is created. Therefore, the microfibers used according to the invention represent an excellent replacement for the foam materials that are otherwise used here and that particularly lack the required restoring forces. A corresponding configuration according to the invention is shown in FIG. 10, for example.

The wound dressing according to the invention may also have a shape that is adapted to the given anatomical circumstances. To this end, it may be constructed in the form of a cuff, for example, which may be rolled up around an arm, a leg, or a joint, or in the form of a bandage that is adapted to the shape of the heel, the elbow joint, or the like.

The wound dressing according to the invention may particularly preferably be adapted such that is suitable for surrounding a surgically attached line. For this purpose, the wound dressing may have at least one slit, for example, enabling the wound dressing to be placed around a line (for example, a drainage tube or catheter) on the patient's body. Such a wound dressing is known, for example, from DE202006005966, held by the present applicant, the contents of which are to be added in their entirety to the disclosure of the present specification. In this context, an arm element, a button, a whipped seam, a weld seam, a perforated stud, or other detachable connecting element may be provided in the distal region of the slit (“predetermined breaking point”) so that the wound dressing may be used in the normal manner or as a slotted compress. In this case, it is particularly important that the wound dressing according to the invention not swell up (lateral distribution) and gain volume when fluid is absorbed because this effect would constrict or block the line surrounded by the dressing. Since the lateral distribution of the fluid prevents the wound treatment article (wound dressing) from increasing in volume, the wound dressing according to the invention is also ideally suited for use in caring for suture wounds (covering) and fistulas (insert).

In this context, it is also preferably provided that the wound dressing has at least one agent designed to suppress bleeding or tendency to bleed.

This agent may be at least one chemically and/or physiologically active agent or active ingredient complex, or at least one physically active element. Such a wound dressing is known, for example, from DE102007030931, held by the present applicant.

For this purpose, the wound dressing may be constructed, for example:
as an absorption material having an essentially flat material section consisting of an absorbent fleece with superabsorbent polymers distributed therein and at least one chemically and/or physiologically active agent or active ingredient complex,
as or in combination with a pressure or compression bandage, particularly in the context of compression therapy for Ulcus cruris venosum,
as a combination of a primary wound dressing having little or no absorbent properties, which has at least one chemically and/or physiologically active agent or active ingredient complex, and a secondary wound dressing arranged peripherally relative to this primary wound dressing and containing superabsorbent polymers, with a diffusion barrier optionally located between the two dressings,
in the form of a bandage pack including one primary dressing with at least one chemically and/or physiologically active agent or active ingredient complex and a wrapping section arranged on the wound dressing, at least portions of which are furnished with superabsorbent polymers, and/or
as an absorption material having a material section extending lengthwise, with the material section having elastically deformable properties, and wherein the material section comprising superabsorbent polymers and optionally having at least one chemically and/or physiologically active agent or active ingredient complex

The chemically and/or physiologically active agent or active ingredient complex is preferably at least one substance or composition having hemostatic properties. These substances are known by the generic term “hemostatic agents.”

The physically effective element may be, for example, a tourniquet, a pressure pad, a pressure bandage, or a compression bandage.

### Definitions

In addition to classic primary and secondary wound dressings, the term “wound treatment article” is also used to refer to wound cleaning cloths, swabs, wound packing items and media, and the like. These items may have absorbing or non-absorbing properties.

The term “exudate” refers to a wound fluid derived from blood plasma due to the inflammatory processes of wound edema. Just as blood is responsible for transporting nutrients and other transmitters, and thus for nourishing various parts of the body, exudate serves in a very similar way to supply the wound bed and the healing processes that are taking place there. In order to be able to perform these many functions properly, it contains a wide range of components, resulting in a specific weight slightly greater than that of water. In this respect, it also differs from transudate, which is caused by non-inflammatory processes and has a considerably lower specific weight and a lower cell and protein content. In addition to providing nutrients for the fibroblasts and epithelial cells, exudate coordinates the various wound healing processes both temporally and spatially with its high content of growth factors and cytokines. These consist mainly of thrombocytes, keratinocytes, macrophages, and fibroblasts. They affect the motility, migration and proliferation of the various cells involved in wound healing. For example, they stimulate migration of cells into the base of the wound as well as supplying the newly formed granulation tissue by angiogenesis. The exudate even assists in cleaning the wound. It contains various serine, cysteine, and aspartate proteases as well as matrix metalloproteinases, whose tightly controlled activity includes breaking down irreversibly damaged tissue, thus preparing the wound bed for subsequent healing phases.

The main components of physiological exudate include salts, glucose, cytokines and growth factors, plasma proteins, proteases (particularly matrix-metalloproteinases), granulocytes, and macrophages.

The term “chemically and/or physiologically active agent or active ingredient complex” is used to designate those active agents or active ingredient complexes that are capable of suppressing bleeding or the tendency to bleed without the need to apply physical force. In this case, the mode of action is a chemical and/or physiological interaction with the wound environment.

In the following, the term “active ingredient complex” will be used to designate not only a complex in the chemical sense, but in particular a combination of ingredients that function synergistically to produce a given effect.

In the following, the term “physically effective element” will be used to refer to an active element that is capable of suppressing bleeding or the tendency to bleed by physical methods, i.e., by the application of pressure, tension, cold, and similar means.

In the following, the term “tourniquet” will be used to refer to an emergency medical means that is capable of stopping arterial blood flow, for example, in a limb, thus preventing unacceptable blood loss from a wound. Indications for such a tourniquet usually include trauma events resulting in injury to at least one artery.

The term “chronic wounds” will be used to refer to wounds that are not primarily attributable to trauma events. While trauma events may have been the original cause of such a wound, the chronic wound is characterized predominately its retarded healing process. Chronic wounds are often associated with little or no bleeding, albeit with frequently heavy exudation.

The term “light bleeding” is used to refer to bleeding that is not arterial in origin, but may be venous or interstitial or capillary, and which in any case is not sufficiently heavy to represent a direct or indirect threat to life.

The term “acutely bleeding wounds” is used to refer to wounds resulting in heavy blood loss. This is usually caused by arterial bleeding, resulting, for example, from the effects of a trauma event. Acutely bleeding wounds may represent a direct or indirect threat to life. For this reason, stopping bleeding is a very high priority in the case of acutely bleeding wounds.

In the following, the term “pressure bandage” is intended to refer to the pressure bandage known from emergency medicine, consisting of a non-absorbent, not overly hard object (pressure pad) with no sharp or hard edges that is applied to a wound that has already been covered and is secured moderately tightly in place by wrapping. The pressure exerted reduces the flow of flow of blood to the affected body part and the blood vessels that have been opened traumatically can be closed again.

The term “poorly absorbent or non-absorbent wound dressing” is used to designate a wound dressing with a low fluid absorption capacity. The total absorption capacity in such cases should be less than 60% by weight, preferably less than 20% by weight, of the dry weight of the dressing. The primary task of such a wound dressing is therefore not to absorb blood or exudates, but rather to release hemostatic agents in the sense of the present invention.

In contrast, the term “compression bandage” is generally used to refer to a bandage that functions in the same way as a pressure bandage but without the application of a pressure pad. In this case, the pressure or compression on the wound is exerted solely by the wrapping medium. In this case, the wrapping material may be elastic.

The term mercerization refers to a finishing process for cotton, which was developed in the mid-19th century by Englishman John Mercer. In mercerization, cotton is immersed in concentrated caustic soda under tension. This causes the fibers to swell, changing their cross-section from a kidney shape to a round shape and shortening the fibers by up to 25%. These structural changes result in a silky, wash-resistant luster, better dyeability, greater strength, improved dimensional stability, and the properties described in the invention. Technically, the properties that are achievable are determined by the merceriziation process used (hot or cold mercerization, dry-in-wet or wet-in-wet methods, rolling or vacuum impregnation) and the effective parameters of product tension, residence times, and caustic soda concentration.

The term “modified natural fibers” refers particularly to the fibers that are known as mercerized fibers and “curled fibers.” These fibers may be straight, wavy, or curled and in the form of staple fibers or continuous tow fibers. A mixture of non-curled fibers whose staple length does not exceed the staple length of the non-curled fibers is advantageous. Fibers with a staple length between 10 mm and 130 mm are considered particularly suitable. The fibers in the form of a fiber-air mixture which are added in mist form to the fiber-air mixture with or without adhesives or lubricants lend themselves well to being sprayed onto an appropriate wound treatment article or parts of a wound treatment article. Of course, these fibers are also suitable for bonding with the corresponding wound treatment articles or parts thereof in a calendering process or with the use of hammer mills. The term “modified natural fibers” also extends to chemical cellulose or cellulose fibers that are obtained using the sulfite process, i.e., by exposing cellulose to sulfites and bisulfites. Particular advantages of the modified natural fibers compared with unmodified natural fibers are their low hemicellulose and resin contents, resulting in a higher white content in the cellulose.

Meltable fibers, which are bonded with the wound treatment article or parts thereof via thermal processes, are especially suitable for bonding with the wound treatment article.

The term “water absorption capacity” (=fluid absorption capacity) is understood to mean the ability to absorb liquid in a given quantity and quality. In this context, “quality” is understood to mean that, when it comes into contact with fibers having good water absorption capacity, the fluid is absorbed quickly by the fibers. Therefore, fibers whose water absorption capacity is lower than their ability to release fluid (demoisturization capability=dehydration capacity) are particularly suitable for the wound treatment articles according to the invention. Accordingly, fibers that have both a good dehydrating capacity and good water absorption capacity are particularly advantageous.

### Drawings and Examples

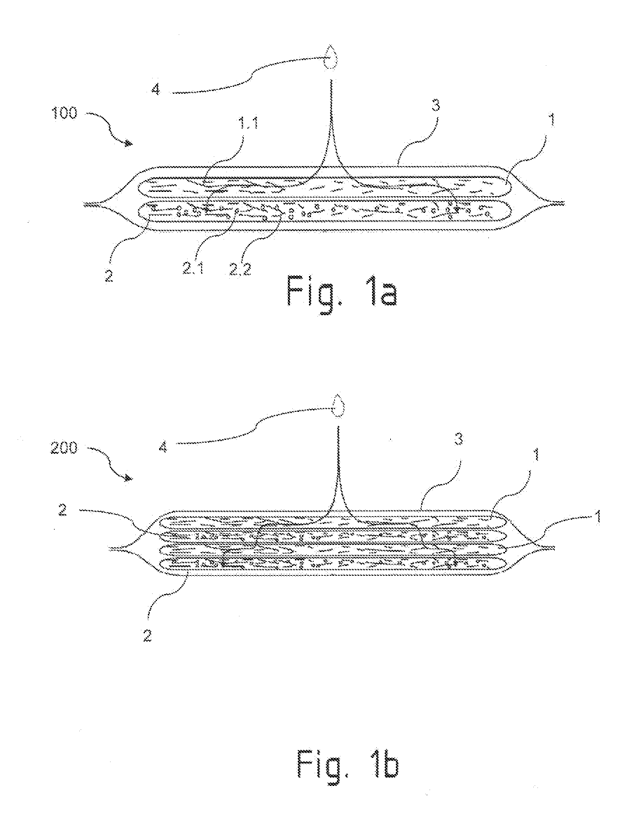

The present invention will be explained in greater detail using the following examples and the discussion of FIGS. 1 to 3. It should be noted that the example and the figures are provided merely for descriptive purposes; they are not intended to be a limitation of the invention in any way.

### Examples

The following example, which is not to be interpreted as a limitation of the present invention, is intended to provide a clearer explanation of the term “curled fibers” as it is understood by the technical community:

### Example 1

Fibrous cellulose material containing individualized, stiffened, curly cellulose fibers and a crosslinking agent, in which the crosslinking agent is selected from C₂-C₈ dialdehydes, C₂-C₈ dialdehyde acid analogs, including at least one aldehyde group, and oligomers of these dialdehydes and dialdehyde acid analogs, with the fibers mentioned above were stiffened chemically in a reaction with a 0.75% to 2.0% molar solution of the crosslinking agent mentioned above relative to a cellulose-anhydroglucose basis and have:
a) an average dry fiber twist count of at least 4.5 twist nodes per millimeter,
b) an average wet fiber twist count of at least about 3.0 twist nodes per millimeter and an average wet fiber twist count of at least 0.5 twist nodes less than the dry fiber twist count,
c) an average isopropyl alcohol retention value of less than 30% and
d) an average water retention value of 28% to 50%.

The fibers cited combine high expansion values when wetted with high wet resilience in absorbent structures.

### Example 2

The mercerized fiber is described in Table 1 with reference to its physical properties and is particularly suitable for use in a wound treatment article since it retains its dehydrating capacity even after multiple cycles in the PFI mill, i.e., with the fibers becoming continuously smaller and under mechanical loading.

TABLE 1

| PFI mill cycles | | 0 | 500 | 1000 | 2000 | 4000 | 8000 |
|---|---|---|---|---|---|---|---|
| Dehydrating capacity | ml | 755 | 767 | 759 | 765 | 757 | 730 |
| Density | g/cm³ | 0.28 | 0.31 | 0.35 | 0.37 | 0.42 | 0.45 |
| Mass | cm³/g | 3.57 | 3.28 | 2.85 | 2.69 | 2.41 | 2.23 |
| Burst index | kPa · m²/g | 0.00 | 0.00 | 0.16 | 0.30 | 0.45 | 1.03 |
| Ductility | N · m/g | 0.6 | 1.0 | 4.1 | 7.2 | 13.6 | 19.7 |
| Breaking length | km | 0.06 | 0.10 | 0.41 | 0.73 | 1.39 | 2.01 |
| Breaking index | mN · m²/g | 2.0 | 3.2 | 5.0 | 7.3 | 10.3 | 16.6 |
| Whiteness | % | 67.7 | 70.1 | 67.3 | 67.9 | 67.0 | 65.6 |

### Example 3

A modified natural fiber obtained from hardwood via a sulfate process and suitable for use in the wound treatment article according to the invention is shown in Table 2.

TABLE 2

| PFI mill cycles | | 0 | 500 | 1000 | 2000 | 4000 | 8000 |
|---|---|---|---|---|---|---|---|
| Dehydrating capacity | ml | 691 | 668 | 622 | 550 | 438 | 253 |
| Density | g/cm³ | 0.38 | 0.45 | 0.46 | 0.52 | 0.57 | 0.63 |
| Mass | cm³/g | 2.67 | 2.23 | 2.16 | 1.93 | 1.74 | 1.58 |
| Burst index | kPa · m²/g | 0.00 | 0.24 | 0.39 | 0.61 | 0.88 | 1.35 |
| Ductility | N · m/g | 4.6 | 9.5 | 11.9 | 17.1 | 22.1 | 30.4 |
| Breaking length | km | 0.47 | 0.97 | 1.21 | 1.74 | 2.25 | 3.10 |
| Breaking index | mN · m²/g | 1.6 | 2.4 | 2.7 | 3.7 | 4.7 | 5.7 |
| Whiteness | % | 82.3 | 81.0 | 79.9 | 79.5 | 78.7 | 77.9 |

Compared to the dehydrating capacity of the modified natural fiber in Table 1 with respect to the PFI cycles, the dehydrating capacity of the modified natural fiber represented in Table 2 diminishes with increasing cycles, but the degree of whiteness is about 10% higher than the values in Table 1.

### Example 4

A modified natural fiber obtained from softwood that retains its mass and porosity and is suitable for use in the wound treatment article according to the invention is shown in Table 3.

TABLE 3

| PFI mill cycles | | 0 | 500 | 1000 | 2000 | 4000 | 8000 |
|---|---|---|---|---|---|---|---|
| Dehydrating capacity | ml | 750 | 745 | 759 | 745 | 694 | 492 |
| Density | g/cm³ | 0.35 | 0.36 | 0.38 | 0.41 | 0.46 | 0.50 |
| Mass | cm³/g | 2.88 | 2.75 | 2.64 | 2.64 | 2.19 | 2.02 |
| Burst index | kPa · m²/g | 0.00 | 0.00 | 0.00 | 0.29 | 0.47 | 0.66 |
| Ductility | N · m/g | 1.0 | 5.3 | 6.1 | 9.1 | 12.0 | 15.9 |
| Breaking length | km | 0.10 | 0.54 | 0.62 | 0.92 | 1.23 | 1.63 |
| Breaking index | mN · m²/g | 2.2 | 3.5 | 3.7 | 4.7 | 4.7 | 4.8 |
| Whiteness | % | 75.6 | 75.9 | 76.0 | 76.0 | 75.2 | 76.0 |

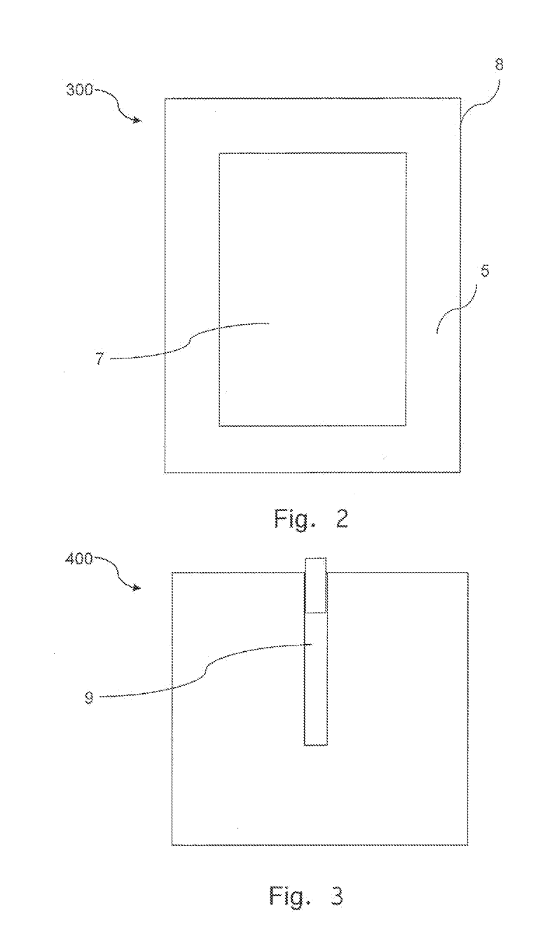

The figures show: FIGS. 1a and 1b show a cross-sectional view of a wound treatment article consisting of plies with mercerized fiber, cellulose, and superabsorbent polymers; FIG. 2 shows a schematic representation of a wound treatment article constructed in the form of a frame; and FIG. 3 shows a schematic representation of a wound treatment article constructed in the form of a slotted compress.

FIG. 1a shows a wound treatment article consisting of two plies 1 and 2, which are surrounded by a common sheath 3. The first ply 1, which preferably represents the ply closest to the wound, consists of modified natural fiber 1.1, in this case a mercerized fiber. A second ply 2 is disposed below the ply 1, and consists of cellulose 2.1, which includes superabsorbent polymers 2.2 dispersed throughout and immobilized in the ply.

The ply 2 may be, for example, an Airlaid as known from the applicant. As is shown in FIG. 1a, a fluid 4 coming into contact with the wound treatment article is diffused laterally over the first ply and then immediately transported vertically into the inside of the ply 2, with the effect that the sheath 3 and ply 1 are redried by the fluid takeup behavior (cellulose 2.1) and fluid absorption (superabsorbent polymers 2.2) of the ply 2.

FIG. 1b shows the same effect as was described previously for FIG. 1a, but here it is even more pronounced, i.e., both the lateral diffusion of the incoming fluid 4 and the redrying of the sheath 3 and the ply 1, which rests on the sheath, are intensified by the double layering of the plies 1 and 2. In addition, the fluid takeup capacity is greater for this wound treatment article than for the wound treatment article shown in 1a.

FIG. 2 shows a wound treatment article 300 with a cutout 7 and a border 8, forming a frame 5. Since the wound treatment article 100 consists of chemically and/or physically modified fibers, the adjacent layers are dried or kept dry. The frame 5 may be used in combination with wound dressings and may be placed under the wound dressing to prevent maceration of the wound edges. In particular, the fibers should be bonded to each other in such way that they form a knitted, woven, or non-woven fabric that may be stamped or cut.

FIG. 3 is an exemplary representation of a wound treatment article 400 having a slit 9 to allow it to be placed around surgically attached tube drainage lines.

I claim:

1. A wound treatment article comprising fibers that have a high dehydrating capacity and at least one wound exudate-absorbing body, wherein the fibers are synthetic fibers or modified natural fibers.

2. The wound treatment article according to claim 1, wherein the fibers are mercerized.

3. The wound treatment article according to claim 1, wherein the fibers have a water absorption capacity that is less than their dehydrating capacity.

4. The wound treatment article according to claim 1, wherein the fibers are extracted from natural materials by chemical processes.

5. The wound treatment article according to claim 4, wherein the natural materials are cotton, cellulose or cellulose-containing materials.

6. The wound treatment article according to claim 5, wherein the fibers obtained from cellulose or cellulose-containing materials have a small hemicellulose and resin component.

7. The wound treatment article according to claim 6, further comprising at least one sheath.

8. The wound treatment article according to claim 7, wherein the sheath is arranged around a section of the wound exudate-absorbing body.

9. The wound treatment article according to claim 8, further comprising superabsorbent polymers.

10. The wound treatment article according to claim 9, further comprising a section having a material selected from the group consisting of soft foam, a modified cellulose, alginates, and a fleece or an air-laid mat or shavings thereof

11. The wound treatment article according to claim 10, wherein the material is carboxymethyl cellulose.

12. The wound treatment article according to claim 11, further comprising at least one fluid-impermeable undergarment protection film section.

13. The wound treatment article according to claim 12, wherein the fluid-impermeable undergarment protection film section is disposed between the sheath and the wound exudate-absorbing body.

14. The wound treatment article according to claim 13, wherein the fluid-impermeable undergarment protection film section is a film on the sheath.

15. The wound treatment article according to claim 1, further comprising at least one slit or cutout.

16. The wound treatment article according to claim 15, wherein the cutout forms the inner part of a frame.

17. A kit for acute, emergency, and military medical use or chronic care comprising the wound treatment article according to claim 1.

18. A method for dressing a wound wherein one applies a wound treatment article according to claim 1 as a wound compress, for cushioning a wound, for placing around surgically attached organ/tube drainage lines, for breaking down a biofilm, and/or for protecting the skin surrounding the wound from maceration.

19. The method according to claim 18, wherein the wound treatment article is disposed in direct contact with the wound so that a section faces the wound and escaping wound exudate passes through said section to the wound exudate-absorbing body.
